(19) United States
(12) Patent Application Publication
(10) Pub. No.: US 2025/0331928 A1
(43) Pub. Date: Oct. 30, 2025

(54) SURGICAL IMAGE GUIDANCE WITH INTEGRATED ELECTROPHYSIOLOGIC MONITORING

(71) Applicant: PRECISION NEUROSCIENCE CORPORATION, New York, NY (US)

(72) Inventors: Benjamin I. RAPOPORT, New York, NY (US); Craig H. Mermel, New York, NY (US); Mark Hettick, New York, NY (US); Adam J. Poole, New York, NY (US); Kyle Reed, New York, NY (US); Ruth Ann Forney, New York, NY (US); Elton Ho, New York, NY (US)

(73) Assignee: PRECISION NEUROSCIENCE CORPORATION, New York, NY (US)

(21) Appl. No.: 19/190,110

(22) Filed: Apr. 25, 2025

Related U.S. Application Data
(60) Provisional application No. 63/638,477, filed on Apr. 25, 2024.

(51) Int. Cl. A61B 34/20; A61B 5/293; A61B 90/00; G06F 3/01

(52) U.S. Cl. CPC A61B 34/20; A61B 5/293; G06F 3/015; A61B 2034/2051; A61B 2034/2055; A61B 2034/2065; A61B 2090/364; A61B 2090/3983; A61B 2562/028; A61B 2562/046

(57) ABSTRACT

A neuronavigation system including a microelectrode array configured to be placed on a brain surface and record neural signals, the microelectrode array comprising a fiducial, and a computer system configured to register a position of the microelectrode array with respect to the brain surface based on the fiducial, receive neural signals from the microelectrode array, and generate a visualization overlaying the received neural signals on a brain image based on the registered position of the microelectrode array for display during a neurosurgical procedure. The neuronavigation system can be used in a variety of different neurosurgical applications, including implanting neural interfaces or locating particular cortical regions.

### PRIORITY

The present application claims priority to U.S. Provisional Patent Application No. 63/638,477, titled SURGICAL IMAGE GUIDANCE WITH INTEGRATED ELECTROPHYSIOLOGIC MONITORING, filed Apr. 25, 2024, which is hereby incorporated by reference herein in its entirety.

### BACKGROUND

Brain-computer interfaces have shown promise as systems for restoring, replacing, and augmenting lost or impaired neurological function in a variety of contexts, including paralysis from stroke and spinal cord injury, blindness, and some forms of cognitive impairment. Multiple innovations over the past several decades have contributed to the potential of these neural interfaces, including advances in the areas of applied neuroscience and multichannel electrophysiology, mathematical and computational approaches to neural decoding, power-efficient custom electronics and the development of application-specific integrated circuits, as well as materials science and device packaging. Nevertheless, the practical impact of such systems remains limited, with only a small number of patients worldwide having received highly customized interfaces through clinical trials.

High bandwidth brain-computer interfaces are being developed to enable the bidirectional communication between the nervous system and external computer systems in order to assist, augment, or replace neurological function lost to disease or injury. A brain-computer interface should be able to accurately decode electrophysiologic signals recorded from individual neurons, or populations of neurons, and correlate such activity with one or more sensory stimuli or intended motor response. For example, such a system can record activity from the primary motor cortex in an animal or a paralyzed human patient and attempt to predict the actual or intended movement in a specific body part; or the system can record activity from the visual cortex and attempt to predict both the location and nature of the stimuli present in the patient's visual field.

Furthermore, brain-penetrating microelectrode arrays have facilitated high-spatial-resolution recordings for brain-computer interfaces, but at the cost of invasiveness and tissue damage that scales with the number of implanted electrodes. In some applications, softer electrodes have been used in brain-penetrating microelectrode arrays; however, it is not yet clear whether such approaches offer a substantially different tradeoff as compared to conventional brain-penetrating electrodes. For this reason, non-penetrating cortical surface microelectrodes represent a potentially attractive alternative and form the basis of the system described here. In practice, electrocorticography (ECoG) has already facilitated capture of high quality signals for effective use in brain-computer interfaces in several applications, including motor and speech neural prostheses. Higher-spatial-resolution micro-electrocorticography (μECoG) therefore represents a promising combination of minimal invasiveness and improved signal quality.

One problem faced in the context of neural interface systems is providing real-time image guidance during the surgical implantation procedure. In particular, superimposing a representation of the functional state of the brain on three-dimensional images of brain structures in the context of real-time surgical image guidance would be highly useful. No high-resolution, real-time solutions exist presently. Functional magnetic resonance imaging (fMRI) provides both functional and structural information about the brain in a manner that permits integrated representation of both types of information. This type of imaging can be helpful in establishing, for example, the locations within the brain of a particular patient responsible for language or motor function. However, both the spatial and temporal resolution of fMRI are relatively coarse (with spatial uncertainty on the order of many millimeters, and time to generate the overlays on the order of many hours). By contrast, the system disclosed here functions in real-time and generates reliable data on a spatial scale of hundreds of microns.

The present disclosure integrates our electrophysiology system, including real-time display capabilities, with surgical image guidance, which is referred to as “neuronavigation.” This is accomplished through real-time, high-resolution, and combined representation of structural and functional status of the brain.

Real-time image guidance is a standard element of many modern surgical procedures. In neurosurgery this capability is often referred to as “neuronavigation,” and denotes the capability of correlating an anatomic location in real space with the corresponding location on three-dimensional imaging, such as brain MRI, typically obtained prior to the procedure (and often used for surgical planning). The dynamic ability to “navigate” surgical instruments in this manner in real time during surgery has led to safer, more accurate, and more precise surgery over the past several decades as the technology has advanced.

Surgical image guidance systems are currently capable of displaying only structural anatomy in real time. The underlying function and state of the anatomic structures being navigated is not reflected in contemporary navigation systems. This is largely because the data imported and displayed by such systems is almost exclusively structural in nature, being derived from volumetric imaging, primarily MRI and computed tomography (CT).

Accordingly, systems and methods for integrating the real-time display of functional (i.e., electrophysiologic) data into the structural (i.e., anatomic) framework of state-of-the-art surgical image guidance (neuronavigation”) would greatly assist practitioners in surgically positioning the neural interfaces, which in turn would improve patient outcomes and the decoding capabilities of the neural interfaces.

### SUMMARY

The present disclosure is directed to systems and methods for providing real-time surgical image guidance using neural interface systems.

In one embodiment, there is provided a neuronavigation system comprising: a microelectrode array configured to be placed on a brain surface and record neural signals, the microelectrode array comprising a fiducial; and a computer system comprising: a processor; and a non-transitory computer-readable medium storing instructions that, when executed by the processor, cause the computer system to: register a position of the microelectrode array with respect to the brain surface based on the fiducial, receive neural signals from the microelectrode array, and generate a visualization overlaying the received neural signals on a brain image based on the registered position of the microelectrode array for display during a neurosurgical procedure.

In one embodiment, there is provided a computer system communicably connectable to a microelectrode array configured to be placed on a brain surface and record neural signals, the microelectrode array comprising a fiducial, the computer system comprising: a processor; and a non-transitory computer-readable medium storing instructions that, when executed by the processor, cause the computer system to: register a position of the microelectrode array with respect to the brain surface based on the fiducial, receive neural signals from the microelectrode array, and generate a visualization overlaying the received neural signals on a brain image based on the registered position of the microelectrode array for display during a neurosurgical procedure.

In one embodiment, there is provided a method for a microelectrode array configured to be placed on a brain surface and record neural signals, the microelectrode array comprising a fiducial, the method comprising: registering, by a computer system, a position of the microelectrode array with respect to the brain surface based on the fiducial, receiving, by the computer system, neural signals recorded by a microelectrode array placed on a brain surface; generating, by the computer system, a visualization overlaying the received neural signals on a brain image based on the registered position of the microelectrode array; and outputting, by the computer system, the visualization for display on a neuronavigation interface during a neurosurgical procedure.

In some embodiments of the systems and methods, the systems and methods can be used in a neurosurgical procedure.

In some embodiments of the systems and methods, the neurosurgical procedure can include implanting the neural interface.

In some embodiments of the systems and methods, the neurosurgical procedure can include removing a tumor, wherein the neural interface is used for identifying a cortical region.

In some embodiments of the systems and methods, the microelectrode array comprises at least 1,000 electrode channels.

In some embodiments of the systems and methods, a position of the microelectrode array is registered relative to the brain image.

In some embodiments, registering the position of the microelectrode array comprises using optical or electromagnetic tracking.

In some embodiments of the systems and methods, the neural signals are processed to provide a spectral analysis on the neural signals to identify oscillatory patterns.

In some embodiments of the systems and methods, the visualization comprises a heatmap overlay indicating levels of neural activity across the brain surface.

In some embodiments of the systems and methods, the visualization is updated in real-time as new neural signals are received from the microelectrode array.

### FIGURES

FIG. 1 depicts a block diagram of a secure neural device data transfer system, in accordance with illustrative embodiments.

FIG. 2 depicts a diagram of a neural device, in accordance with illustrative embodiments.

FIG. 3 depicts a diagram of a thin-film, microelectrode array neural device and implantation method, in accordance with illustrative embodiments.

FIG. 4 depicts a patient management interface for the neuronavigation system, in accordance with an embodiment of the present disclosure.

FIG. 5 depicts a surgical approach and planning interface for the neuronavigation system, in accordance with an embodiment of the present disclosure.

FIG. 6 depicts an interface for registering the electrode arrays for the neuronavigation system, in accordance with an embodiment of the present disclosure.

FIG. 7 depicts a first screen showing preoperative brain imaging data for the neuronavigation system, in accordance with an embodiment of the present disclosure.

FIG. 8 depicts a second screen showing preoperative brain imaging data for the neuronavigation system for the neuronavigation system, in accordance with an embodiment of the present disclosure.

FIG. 9 depicts a third screen showing preoperative brain imaging data for the neuronavigation system for the neuronavigation system, in accordance with an embodiment of the present disclosure.

FIG. 10 depicts a visualization of the registered array placement for the neuronavigation system, in accordance with an embodiment of the present disclosure.

FIG. 11 depicts intraoperative brain imaging data for the neuronavigation system, in accordance with an embodiment of the present disclosure.

FIG. 12 depicts a visualization merging merge the preoperative brain image data with the intraoperative brain scan for the neuronavigation system, in accordance with an embodiment of the present disclosure.

FIG. 13 depicts an array adjustment interface for the neuronavigation system, in accordance with an embodiment of the present disclosure.

FIG. 14 depicts a dot plot visualization for the neuronavigation system, in accordance with an embodiment of the present disclosure.

FIG. 15 depicts a dot plot visualization interface grouped by electrode arrays corresponding to different decoding tasks and anatomical regions for the neuronavigation system, wherein in accordance with an embodiment of the present disclosure.

FIG. 16 depicts a sparkline visualization for the neuronavigation system, in accordance with an embodiment of the present disclosure.

FIG. 17 depicts an electrode channel selection interface for the neuronavigation system, wherein a column of electrodes is selected, in accordance with an embodiment of the present disclosure.

FIG. 18 depicts the electrode channel selection interface for the neuronavigation system, wherein a row of electrodes is selected, in accordance with an embodiment of the present disclosure.

FIG. 19 depicts the electrode channel selection interface for the neuronavigation system, wherein individual electrodes are selected, in accordance with an embodiment of the present disclosure.

FIG. 20 depicts the electrode channel selection interface for the neuronavigation system, wherein a sparse grid of electrodes is selected, in accordance with an embodiment of the present disclosure.

FIG. 21 depicts the electrode channel selection interface for the neuronavigation system, wherein a rectangular group of electrodes is selected, in accordance with an embodiment of the present disclosure.

FIG. 22 depicts the sparkline visualization used for surgical plan management by allowing for surgeons to select, record, and track neural signal activity across different electrode arrays separately for the neuronavigation system, in accordance with an embodiment of the present disclosure.

FIG. 23 depicts an interface for visualizing the neural data in a digital and/or analog manner for the neuronavigation system, in accordance with an embodiment of the present disclosure.

FIG. 24 depicts a spectrogram visualization for the neuronavigation system, in accordance with an embodiment of the present disclosure.

FIG. 25 depicts a data replay interface for the neuronavigation system, in accordance with an embodiment of the present disclosure.

### DETAILED DESCRIPTION

The present disclosure is generally directed to surgical systems and methods for enabling real-time, intraoperative neuronavigation. In particular, the present disclosure is directed to providing visualizations and interfaces for assisting surgeons and other surgical staff in implanting and placing neural devices.

### Neural Device Systems

Conventional BCI/neural devices typically include electrode arrays that penetrate a subject's brain to sense and/or stimulate the brain. However, the present disclosure is directed to the use of non-penetrating BCI devices, i.e., BCI devices having electrode arrays that do not penetrate the cortical surface. Such non-penetrating BCI devices are minimally invasive and minimize the amount of impact on the subject's cortical tissue. BCI devices can sense and record brain activity, receive instructions for stimulating the subject's brain, and otherwise interact with a subject's brain as generally described herein.

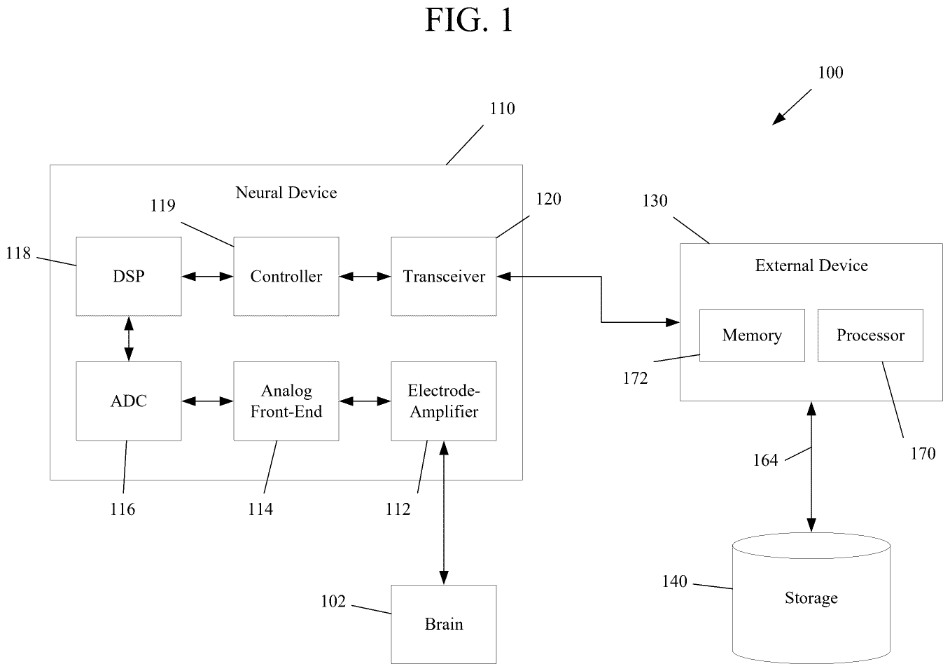

Referring now to FIGS. 1-3, there is shown a diagram of an illustrative system 100 including a neural device 110 that is communicatively coupled to an external device 130. The external device 130 can include any device to which the neural device 110 can be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 130 can include a processor 170 and a memory 172. In some embodiments, the external device 130 can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure).

In some non-limiting embodiments, the electrode array 180 can have about 100 electrodes, about 200 electrodes, about 300 electrodes, about 400 electrodes, about 500 electrodes, about 600 electrodes, about 700 electrodes, about 800 electrodes, about 900 electrodes, about 1,000 electrodes, about 1,500 electrodes, about 2,000 electrodes, about 2,500 electrodes, about 3,000 electrodes, about 4,000 electrodes, about 5,000 electrodes, or ranges between any two of these values, including endpoints. In one embodiment, the electrode array 180 can have 1,000 or more electrodes. In one illustrative embodiment, the electrode array 180 can have 1,024 electrodes. In some embodiments, the electrode array 180 of the neural device 110 can have electrodes that are sufficiently small and spaced at sufficiently small distances in order to define a high-density electrode array 180 that can, accordingly, capture high resolution electrocortical data. Such high-resolution data can be used to resolve electrographic features that can otherwise not be identified using lower resolution electrode arrays. In some embodiments, the electrodes of the electrode array 180 can be from about 10 μm to about 500 μm in width. In one illustrative embodiment, the electrodes of the electrode array 180 can be about 50 μm in width. In some embodiments, the electrodes of the electrode array 180 can be spaced by about 200 μm (i.e., 0.2 mm) to about 3,000 μm (i.e., 3 mm). In illustrative one embodiment, adjacent electrodes of the electrode array 180 can be spaced by about 400 μm.

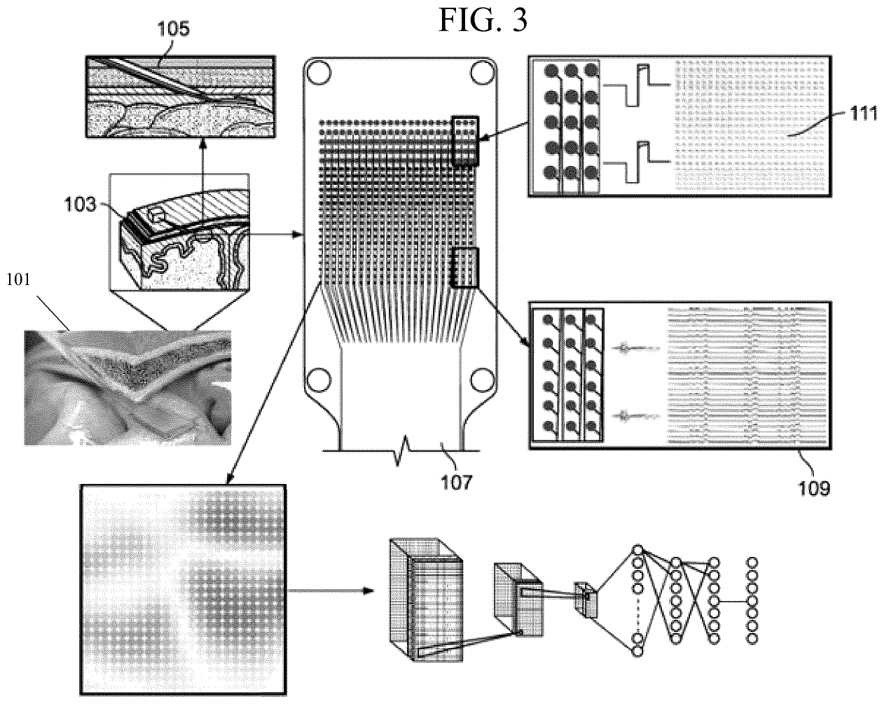

The neural device 110 can further include a flexible substrate 212 supporting the electrode array 180 and/or other components of the neural device 110, as shown in FIG. 3. In some embodiments, the flexible substrate 212 can be flexible enough to permit the electrode array 180 to be inserted through an osteotomy into the subdural space 204, then along the cortical surface.

The neural device 110 can include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 can include an electrode array 180, such as is described below, that is able to physically interface with the brain 102 of the subject in order to sense brain signals and/or apply electrical signals thereto. The analog front-end stage 114 can be configured, amplify signals that are sensed from or applied to the brain 102, perform conditioning of the sensed or applied analog signals, perform analog filtering, and so on. The front-end stage 114 can include, for example, one or more application-specific integrated circuits (ASICs) or other electronics. The ADC stage 116 can be configured to convert received analog signals to digital signals. The DSP stage 118 can be configured to perform various DSP techniques, including multiplexing of digital signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the subject.

In some embodiments, the neural device 110 can include a controller 119 that is configured to perform various functions, including compressing electrophysiologic data generated by the electrode array 180. In various embodiments, the controller 119 can include hardware, software, firmware, or various combinations thereof that are operable to execute the functions described below. In one embodiment, the controller 119 can include a processor (e.g., a microprocessor) executing instructions stored in a memory. In another embodiment, the controller 119 can include a field-programmable gate array (FPGA) or application-specific integrated circuit (A SIC).

In various embodiments, the stages of the neural device 110 can provide unidirectional or bidirectional communications (as indicated in FIG. 1) by and between the neural device 110 and the external device 130. In various embodiments, one or more of the stages can operate in a serial or parallel manner with other stages of the system 100. It can further be noted that the depicted architecture for the system 100 is simply intended for illustrative purposes and that the system 100 can be arranged differently (i.e., components or stages can be connected in different manners) or include additional components or stages.

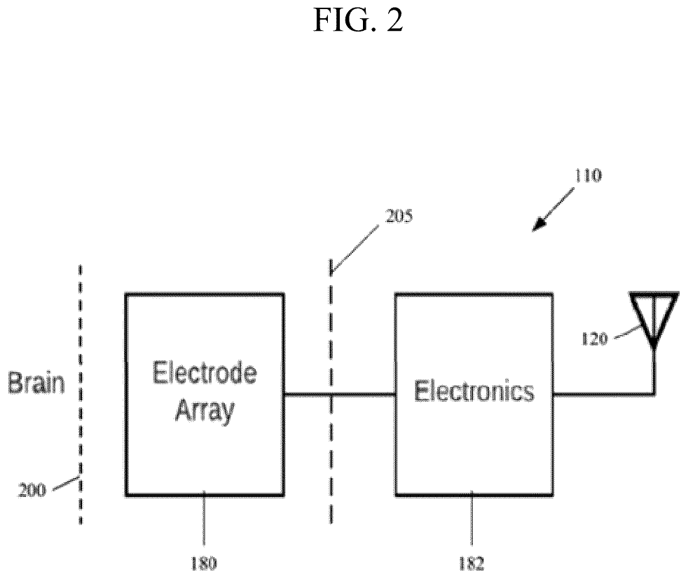

In some embodiments, the neural device 110 described above can include a brain implant, such as is shown in FIG. 2. The neural device 110 can be a biomedical device configured to study, investigate, diagnose, treat, and/or augment brain activity. In some embodiments, the neural device 110 can be positioned between the brain 200 and the scalp or between the brain and the dura 205 in the subdural space 204, as shown in FIG. 3. The neural device 110 can include an electrode array 180 (which can be a component of or coupled to the electrode-amplifier stage 112 described above) that is configured to record and/or stimulate an area of the brain 200. The electrode array 180 can be connected to an electronics hub 182 (which can include one or more of the electrode-amplifier stage 112, analog front-end stage 114, ADC stage 116, and DSP stage 118) that is configured to transmit via wireless or wired transceiver 120 to the external device 130 (in some cases, referred to as a “receiver”).

The electrode array 180 can include non-penetrating cortical surface microelectrodes (i.e., the electrode array 180 does not penetrate the brain 200). Accordingly, the neural device 110 can provide a high spatial resolution, with minimal invasiveness and improved signal quality. The minimal invasiveness of the electrode array 180 is beneficial because it allows the neural device 110 to be used with larger population of subjects than conventional brain implants, thereby expanding the application of the neural device 110 and allowing more individuals to benefit from brain-computer interface technologies. Furthermore, the surgical procedures for implanting the neural devices 110 are minimally invasive, reversible, and avoid damaging neural tissue. In some embodiments, the electrode array 180 can be a high-density microelectrode array that provides smaller features and improved spatial resolution relative to conventional neural implants.

In some embodiments, the neural device 110 includes an electrode array configured to stimulate or record from neural tissue adjacent to the electrode array, and an integrated circuit in electrical communication with the electrode array, the integrated circuit having an analog-to-digital converter (ADC) producing digitized electrical signal output. In some embodiments, the ADC or other electronic components of the neural device 110 can include an encryption module, such as is described below. The neural device 110 can also include a wireless transmitter (e.g., the transceiver 120) communicatively coupled to the integrated circuit or the encryption module and an external device 130. The neural device 110 can also include, for example, control logic for operating the integrated circuit or electrode array 180, memory for storing recordings from the electrode array, and a power management unit for providing power to the integrated circuit or electrode array 180.

Referring now to FIG. 3, there is shown a diagram of an illustrative embodiment of a neural device 110. In this embodiment, the neural device 110 comprises an electrode array 180 comprising nonpenetrating microelectrodes. As generally described above, the neural device 110 is configured for minimally invasive subdural implantation using a cranial micro-slit technique, i.e., is inserted into the subdural space 204 between the dura 205 and the surface of the subject's brain 200. Further, the microelectrodes of the electrode array 180 can be arranged in a variety of different configurations and can vary in size. In this particular example, the electrode array 180 includes a first group 190 of electrodes (e.g., 200 μm microelectrodes) and a second group 192 of electrodes (e.g., 20 μm microelectrodes). Further, example stimulation waveforms in connection with the first group 190 of electrodes and the resulting post-stimulus activity recorded over the entire array is depicted for illustrative purposes. Still further, example traces from recorded neural activity recorded by the second group 192 of electrodes are likewise illustrated. In this example, the electrode array 180 provides multichannel data that can be used in a variety of electrophysiologic paradigms to perform neural recording of both spontaneous and stimulus-evoked neural activity as well as decoding and focal stimulation of neural activity across a variety of functional brain regions.

Additional information regarding brain-computer interfaces described herein can be found in Ho et al., The Layer 7 Cortical Interface: A Scalable and Minimally Invasive Brain-Computer Interface Platform, bioRxiv 2022.01.02.474656; doi: https://doi.org/10.1101/2022.01.02.474656, which is hereby incorporated by reference herein in its entirety. Additional information regarding high-resolution, thin-film surface electrode arrays can further be found in U.S. Patent Application Publication No. 2024/0115178, titled SYSTEMS AND METHODS FOR NEURAL INTERFACES, filed Oct. 17, 2023, which is hereby incorporated by reference herein in its entirety.

Surgical Image Guidance with Integrated Electrophysiologic Monitoring

As noted above, the present disclosure is generally directed to high-resolution, thin-film surface electrode arrays, which can be used for detecting and displaying in real-time electrophysiologic activity of the brain. By correlating the precise location of these electrode arrays in space with the corresponding positions in three-dimensional imaging studies (such as MRI and CT studies obtained prior to the surgical procedure being performed), it is possible to generate composite renderings overlaying the functional activity of the brain on images of the structural anatomy. Such surgical image guidance can be useful, for example, in the context of implanting BCIs, as described throughout. However, the neuronavigation systems and techniques described herein are not limited solely to implanting B CIs and can generally be used in a variety of different neurosurgery applications. High-definition electrophysiology rendered together with structural imaging allows for precise localization of functional brain areas that is not possible to delineate using structural anatomy alone, which is generally useful in neurosurgery applications. For example, locating cortical regions involved in language, motor, or other functions is useful when removing a brain tumor or a focus of seizure activity.

The ability to integrate the functional and structural data depends in part on being able to reliably locate certain key calibration points on the electrode array in order to locate the position and orientation of the electrode array and then infer the position of each of the electrodes on the array relative to these calibration points and surrounding anatomic structures. General techniques for calibrating surgical image guidance have been used in the context of other surgical procedures. However, neural interface systems currently lack the ability to integrate real-time functional state of the brain into an image guidance platform using calibration points built into the electrode array, as described herein.

The present disclosure relates to systems and methods for implementing real-time surgical image guidance in a neural interface system 100. By integrating high-resolution electrophysiological data with neuronavigation capabilities, the system can provide surgeons with enhanced visualization and decision-making tools during neurosurgical procedures. In some cases, the system can combine data from a high-density electrode array with anatomical imaging to create an augmented reality view of functional brain activity overlaid on structural anatomy. This integration can allow for more precise localization of eloquent cortical areas and functional boundaries during procedures such as tumor resection or epilepsy surgery.

The system can enable real-time tracking of electrode array placement and visualization of neural signals directly within the surgical field of view. In some implementations, the neuronavigation interface can display functional mapping data alongside traditional anatomical landmarks and surgical planning information. By providing a unified interface that merges electrophysiological recordings with neuronavigation, the system can streamline intraoperative workflows and enhance communication between surgeons and neurophysiologists. The real-time nature of the integrated display can allow for dynamic updates as the surgery progresses and brain structures shift.

In some cases, the system can incorporate machine learning algorithms to process the high-dimensional neural data and extract clinically relevant features for display. This processing can occur in real-time to provide actionable insights to the surgical team throughout the procedure. The integration of neural interface capabilities with surgical navigation can enable new possibilities for precision and personalization in neurosurgery. By providing surgeons with a more comprehensive view of both brain structure and function, the system can support improved surgical outcomes and reduced risk of neurological deficits.

In some cases, the system can include a neuronavigation system for providing real-time imaging and guidance during neurosurgical procedures. The described systems, processes, and techniques for neuronavigation system integrated with a high-density electrode array can be implemented within the context of the neural interface system 100 shown in FIGS. 1-3 and described above, for example.

The neuronavigation system can include imaging capabilities for capturing preoperative and intraoperative images of a patient's brain anatomy. Initially, a brain image can be obtained prior to surgery to map the patient's brain structures. The brain image can be a high-resolution MRI or CT scan that provides detailed anatomical information. During the surgical procedure, intraoperative imaging can be performed to account for brain shift and update the navigation reference frame. For example, a sagittal brain scan can be acquired during surgery. The sagittal brain scan can provide an updated view of the brain anatomy after the craniotomy has been performed. The neuronavigation system can register and merge the preoperative and intraoperative imaging data to create an integrated 3D model of the patient's brain. This model can serve as the basis for surgical planning and real-time navigation.

In some implementations, the neuronavigation system can include optical or electromagnetic tracking capabilities to monitor the position of surgical instruments and the patient's head in 3D space. Fiducial markers placed on the patient and instruments can allow their locations to be precisely tracked relative to the brain imaging data.

The system can provide a graphical user interface for surgeons to interact with the 3D brain model and plan surgical trajectories. In various implementations, the interface can allow marking of targets, definition of safe corridors, and visualization of critical structures to avoid. During the procedure, the neuronavigation system can provide real-time guidance by displaying the position of tracked instruments overlaid on the registered brain images. This can allow surgeons to navigate precisely to target locations while avoiding eloquent areas.

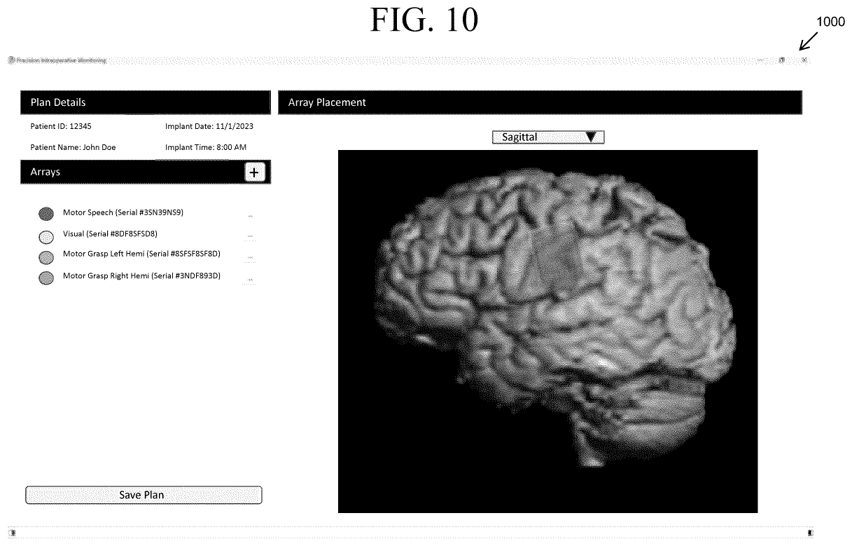

In some cases, the electrode array can be integrated with the neuronavigation system to enable visualization of array placement and electrophysiological data in the context of brain anatomy. The array location can be tracked and displayed on the neuronavigation interface in real-time as it is positioned on the cortical surface, as shown in FIG. 10. As described herein, the array placement visualization 1000 shown in FIG. 10 can be determined by registering the position and orientation of the electrode arrays 180 with respect to the patient's cortical surface using the fiducials disposed on the electrode arrays 180. The combined neuronavigation and electrode array system can provide surgeons with multimodal information integrating structural and functional data. This integration can support more precise targeting of brain regions and identification of functional boundaries during procedures such as tumor resection.

In some cases, the system can include a high-resolution electrode array 180 for recording neural signals from the brain surface, such as is described above. As generally described above, the electrode array 180 can be configured to interface directly with cortical tissue to detect electrical activity. The electrode array 180 can comprise a flexible substrate that allows the array to conform to the curvature of the brain surface. This flexibility can enable close contact between the electrodes and neural tissue, potentially improving signal quality and spatial resolution. In some implementations, the electrode array 180 can include various different configurations and numbers of recording channels arranged in a high-density configuration. In one illustrative embodiment, the electrode array 180 can include 1,024 individual electrodes, i.e., recording channels. The large number of channels can allow for detailed mapping of neural activity across the covered brain region. In some embodiments, the electrode array 180 can present a compact form factor that enables coverage of specific cortical areas of interest, while minimizing the footprint on the brain surface. The high channel count and density of the electrode array 180 can enable recording of neural signals with high spatial and temporal resolution. This detailed electrophysiological data can be integrated with structural information from the brain image and the sagittal brain scan to provide a comprehensive view of brain structure and function during neurosurgical procedures.

In some cases, the electrode array 180 can be designed for temporary placement during acute recording sessions, such as intraoperative monitoring during tumor resection. The array can be positioned on the cortical surface to map functional areas adjacent to the surgical site. Further, the flexible nature of the electrode array 180 can allow it to maintain consistent contact with the brain surface even as the cortex deforms or shifts during the surgical procedure. This can help ensure stable signal quality throughout the recording session.

In some cases, the system can employ various signal processing techniques and visualization methods to interpret and display neural activity data recorded by the electrode array. These techniques can allow for real-time analysis and presentation of complex electrophysiological signals in formats that are intuitive and actionable for the surgical team.

In some cases, the system can implement a workflow for using the integrated neuronavigation and neural recording capabilities during surgical procedures. This workflow can involve coordination between the neurosurgeon and a neuronavigation representative to plan and execute the procedure.

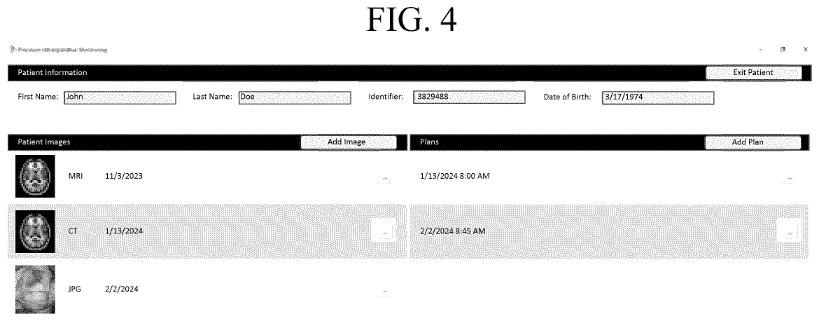

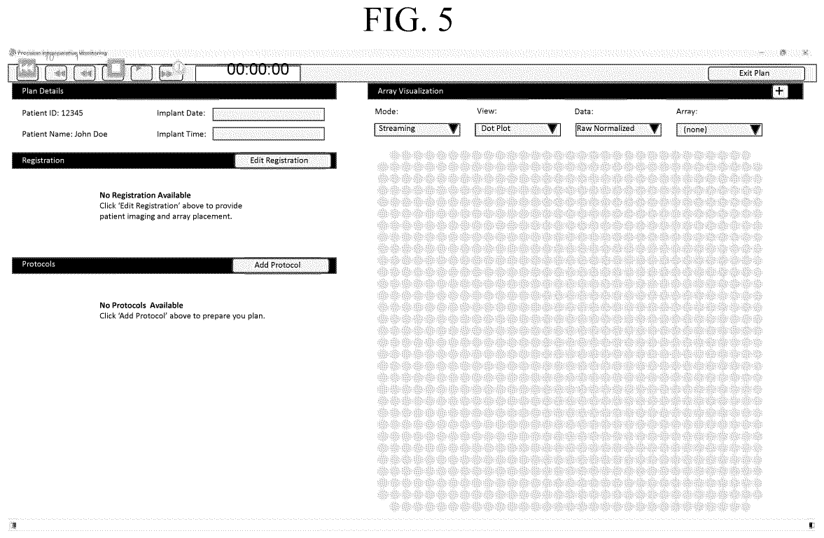

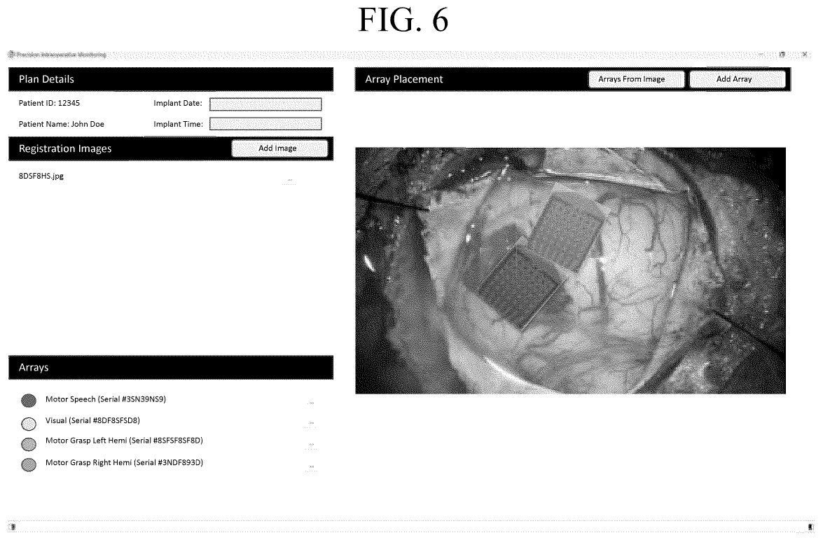

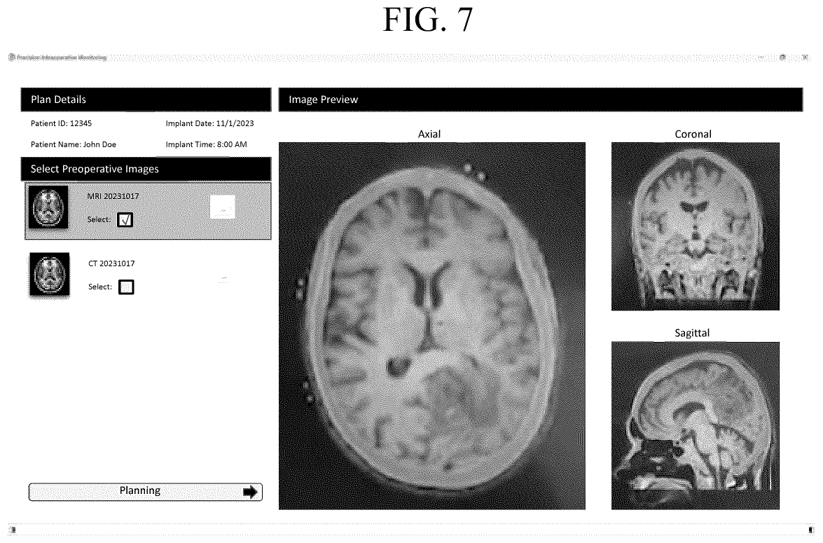

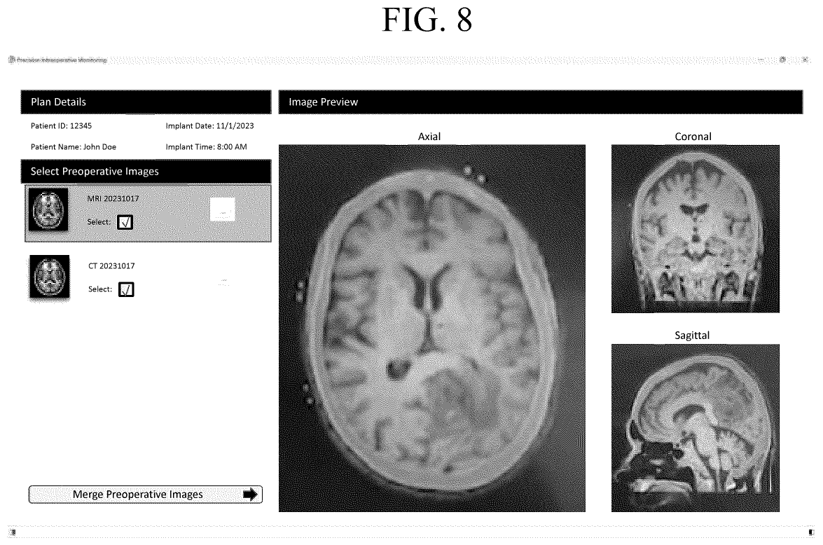

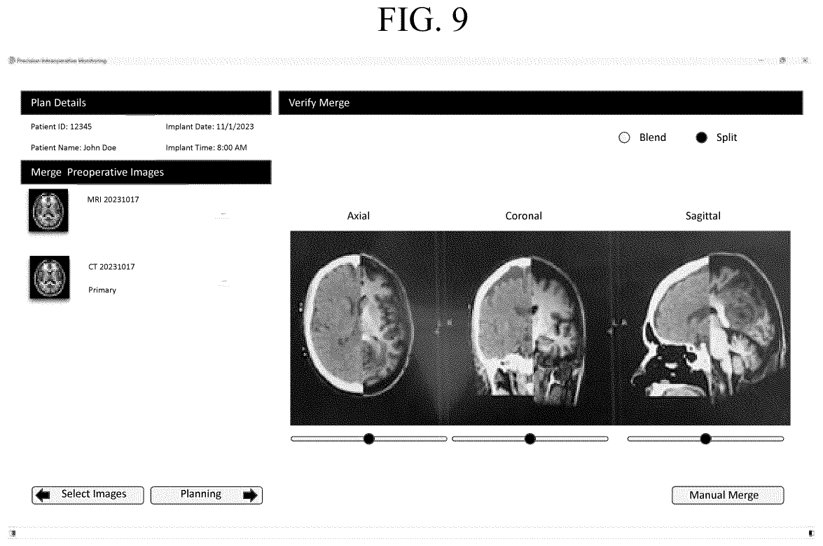

As illustrated in FIGS. 4 and 5, the workflow for using the neuronavigation system described herein can begin with identifying the patient using patient management features (FIG. 4) and establishing the surgical approach and plan (FIG. 5). This initial planning phase can involve reviewing preoperative imaging data (FIGS. 7-9) and determining optimal placement locations for the electrode array. Following the initial planning, the neuronavigation system can be calibrated to establish accurate spatial registration (FIG. 6) between the patient's anatomy and the imaging data. The neurosurgeon and representative can then work together to determine specific placement targets for the electrode array based on the surgical objectives and anatomical considerations.

In various embodiments, the position of the electrode array 180 can be registered relative to the brain image using optical and/or electromagnetic tracking techniques. In one embodiment, the electrode array 180 can include fiducials that can be optically identified (e.g., by cameras or image sensors located within the operating room) and used to register the position of the electrode array 180 with respect to the patient's anatomy and/or within the surgical environment. In various embodiments, the fiducials can include radiopaque markings, QR codes, ArU co markers, or any other type of fiducial that can be visually identified by the imaging system. Further, the fiducials can be used to identify and register multiple different electrode arrays 180 along the cortical surface, as shown in FIG. 6. Further, the electrode arrays 180 can be registered with respect to different cortical surface areas and/or decoding tasks, as also shown in FIG. 6. In another embodiment, the electrode array 180 electromagnetic sensors that can be utilized to track the position of the electrode array 180 within an electromagnetic tracking system.

The system can support multiple visualization modes to represent neural activity patterns. For example, a dot plot visualization can be used to display signal intensity across multiple electrode channels. In this visualization, each electrode can be represented by a colored dot, with the color indicating the level of neural activity detected at that location. The dot plot can provide a spatial map of activity patterns across the entire electrode array. In some implementations, the system can generate spectrogram visualizations to represent the frequency content of neural signals over time. For example, the system can include a spectrogram display where the vertical axis can represent frequency, while the horizontal axis can represent time. Signal power at different frequencies can be indicated by color intensity, allowing for identification of oscillatory patterns or changes in spectral content during the recording. In some implementations, the system can provide heatmap visualizations to represent spatial patterns of neural activity. Heatmaps can use color gradients to indicate signal intensity or other derived metrics across the electrode array. This visualization mode can be particularly useful for identifying regions of elevated activity or mapping functional boundaries on the cortical surface. In some implementations, the system can provide playback and time-synchronized visualization of neural data. For example, recorded neural activity can be replayed and visualized alongside other relevant data streams. This capability can enable retrospective analysis of specific events or time periods during the surgical procedure. The system can employ various signal processing algorithms to extract meaningful features from the raw neural recordings. These can include spectral analysis techniques to quantify oscillatory activity in different frequency bands, connectivity measures to assess functional interactions between brain regions, and dimensionality reduction methods to identify dominant patterns in the high-dimensional data.

In some implementations, the system can use adaptive thresholding or statistical techniques to detect significant changes in neural activity patterns. These detected events can be highlighted in the visualizations to draw attention to potentially relevant shifts in brain state or responses to surgical manipulations. The signal processing and visualization components can be designed to operate in real-time, allowing for immediate feedback during the surgical procedure. Low-latency processing pipelines and efficient rendering techniques can be employed to ensure that visualizations remain responsive and up-to-date as new neural data is continuously acquired. In some cases, the system can provide options for customizing visualizations based on user preferences or specific clinical needs. This can include adjustable color scales, selectable frequency bands of interest, or the ability to focus on particular spatial regions or subsets of electrodes. The integration of advanced signal processing and intuitive visualization methods can enhance the utility of high-resolution neural recordings during neurosurgical procedures. By transforming complex electrophysiological data into clear, interpretable displays, the system can support more informed decision-making and precise functional mapping in the operating room.

In some cases, the system can integrate neural activity data recorded by the electrode array with anatomical imaging displayed by the neuronavigation system. This integration can allow for real-time visualization of functional boundaries overlaid on brain images during neurosurgical procedures.

In some cases, electrode array placement can be planned and visualized within the neuronavigation interface. For example, the neuronavigation interface can display various overlays representing the planned position of the electrode array over the brain image within the interface. In some implementations, the system can allow the surgeon to adjust the planned array position by manipulating the overlay on the brain image.

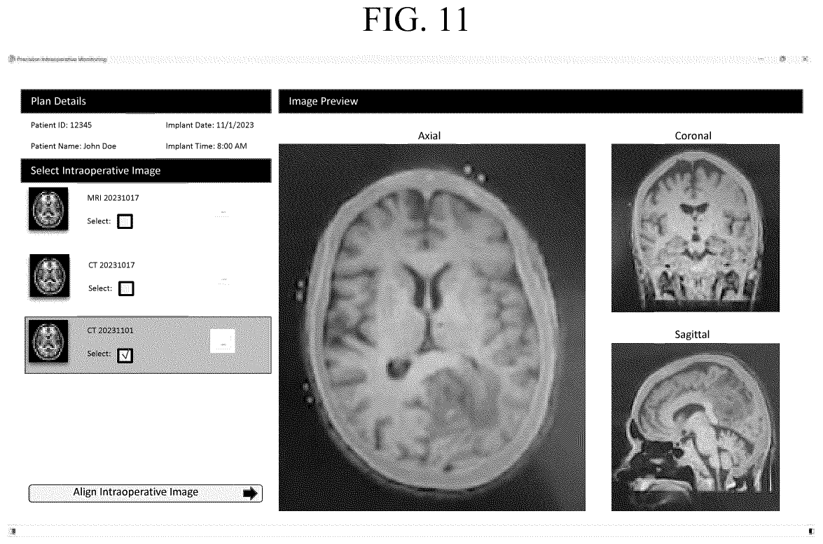

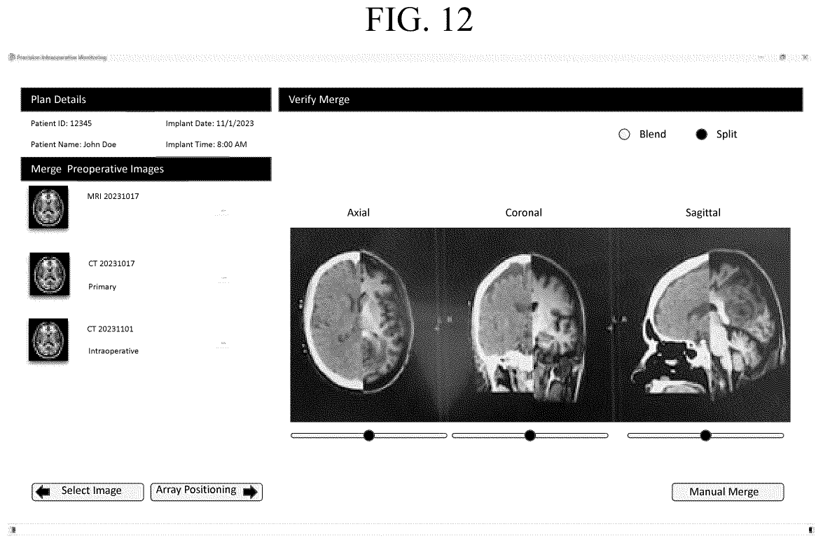

The process of registering the electrode array position with anatomical structures can involve several steps. Initially, the neuronavigation system can use preoperative imaging such as the brain image to create a 3D model of the patient's brain anatomy. During the surgical procedure, intraoperative imaging such as a sagittal brain scan can be acquired to account for brain shift and update the anatomical reference frame. For example, as shown in FIG. 12, the system 100 can merge the preoperative brain image (FIGS. 7-9) with the intraoperative brain scan (FIG. 11). This merged imaging data can provide an updated anatomical reference for registering the electrode array position. Once the electrode array is placed on the cortical surface, the system can use various methods to determine the array's position relative to the brain anatomy. In some cases, optical or electromagnetic tracking can be used to localize the array. Alternatively, intraoperative imaging such as the sagittal brain scan can be used to visualize the array's position directly.

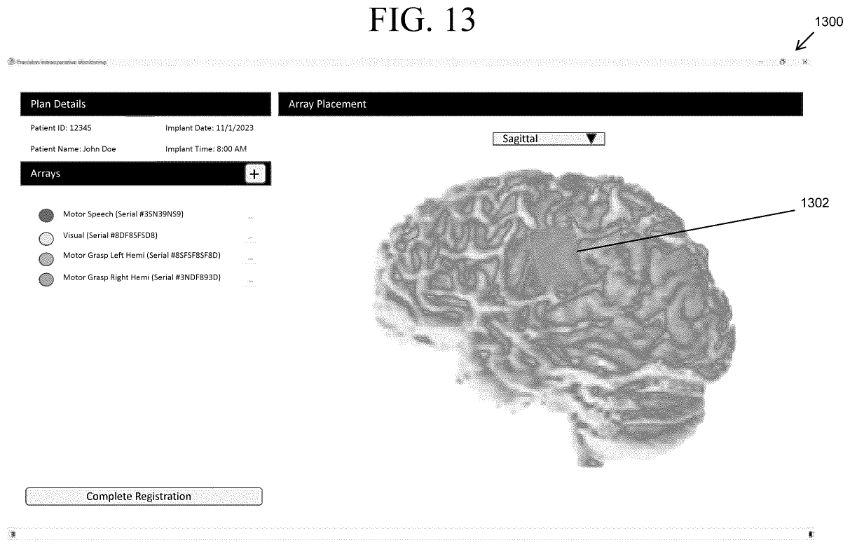

In some embodiments, the electrode array position can be fine-tuned within the neuronavigation interface. For example, FIG. 13 depicts an illustrative array adjustment interface 1300 that shows a brain image with a rectangular overlay 1302 representing the electrode array 180, along with controls for adjusting the array's position and orientation. In some implementations, once the electrode array position is confirmed, the system can allow the array position to be “locked” on the neuronavigation screen. This locking feature can ensure that the visualized array position remains stable even if the physical array shifts slightly during the procedure.

The integration of neural data with neuronavigation can enable real-time visualization of functional boundaries overlaid on the brain images. As the electrode array records neural activity, the system can process the data to identify regions of elevated activity or functional importance. These identified regions can be displayed as color-coded overlays or contour lines superimposed on the anatomical imaging within the neuronavigation interface. In some cases, the system can update the functional boundary visualization in real-time as new neural data is acquired. This dynamic display can allow surgeons to track changes in functional activity patterns throughout the surgical procedure, potentially informing decisions about resection boundaries or stimulation targets. The integrated visualization of neural activity and brain anatomy can provide surgeons with a comprehensive view of both structure and function during neurosurgical procedures. This multimodal information can support more precise targeting of brain regions and identification of eloquent areas to be preserved during resection.

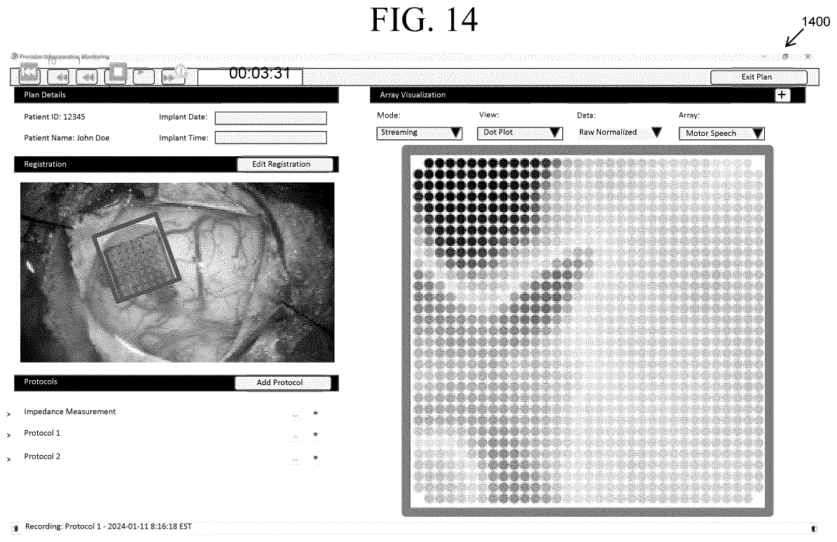

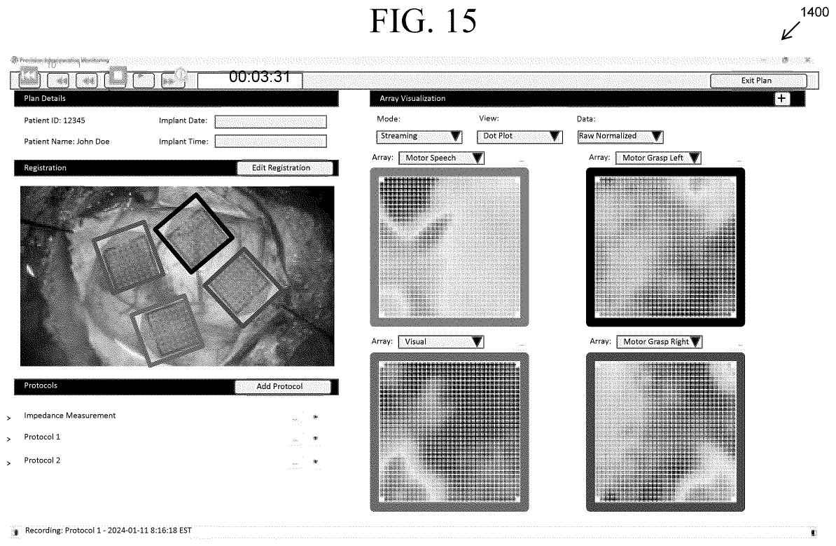

In some cases, the system can provide real-time visualization of functional boundaries based on neural activity data recorded by the high-density electrode array. This capability can allow surgeons to identify eloquent cortical areas and guide surgical decision-making during procedures such as tumor resection or epilepsy surgery. The system can employ various signal processing techniques to analyze the high-dimensional neural data in real-time and extract features indicative of functional boundaries. These techniques can include spectral analysis, connectivity measures, and statistical methods to detect significant changes in neural activity patterns across the electrode array. Based on this analysis, the system can generate dynamic visualizations that highlight regions of elevated activity or functional importance. For example, FIGS. 14 and 15 depict an illustrative dot plot representation 1400 that can be used to display neural activity patterns across the electrode array. In this visualization, each electrode channel can be represented by a colored dot, with the color intensity indicating the level of neural activity detected at that location. The dot plot visualization can be updated in real-time as new neural data is acquired, allowing for continuous monitoring of functional activity throughout the surgical procedure. The spatial arrangement of the dots can correspond to the physical layout of the electrode array, providing an intuitive mapping between the visualization and the cortical surface.

In some cases, the system can use thresholding or clustering algorithms to automatically identify and delineate functional boundaries within the neural activity patterns. These boundaries can be overlaid on the dot plot visualization as contour lines or highlighted regions, drawing attention to areas of potential functional importance. The functional boundary visualization can be integrated with the neuronavigation interface, allowing surgeons to view the real-time neural activity patterns in the context of the patient's brain anatomy. This integration can enable more precise correlation between functional boundaries and anatomical landmarks or surgical targets. The system can provide options for customizing the visualization parameters to suit specific clinical needs. For example, surgeons can be able to adjust color scales, set activity thresholds for boundary detection, or focus on particular frequency bands of interest in the neural signals. In some implementations, the system can support multiple visualization modes for representing functional boundaries. These can include heatmaps, topographic maps, or 3D surface renderings that provide alternative views of the neural activity patterns and functional organization. The real-time nature of the functional boundary visualization can allow surgeons to monitor changes in cortical activity patterns throughout the procedure. This dynamic information can be particularly valuable for tracking the effects of surgical manipulations, such as temporary lesions or stimulation, on functional organization.

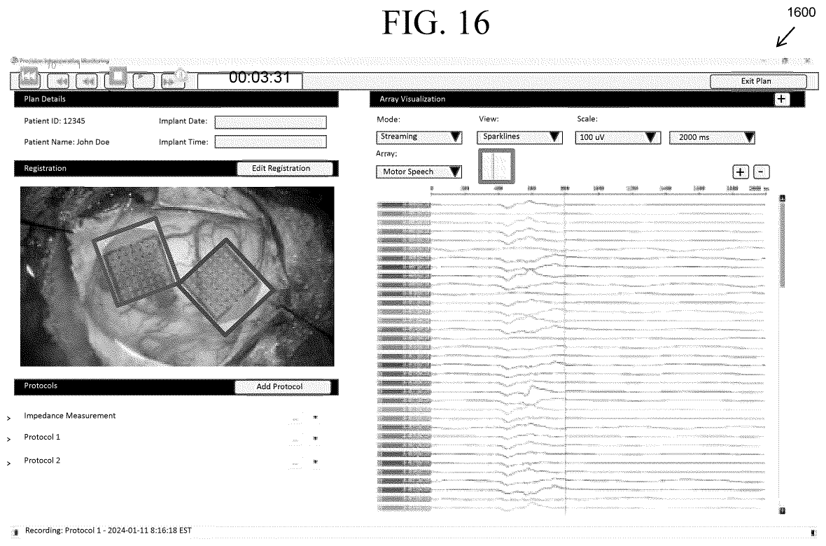

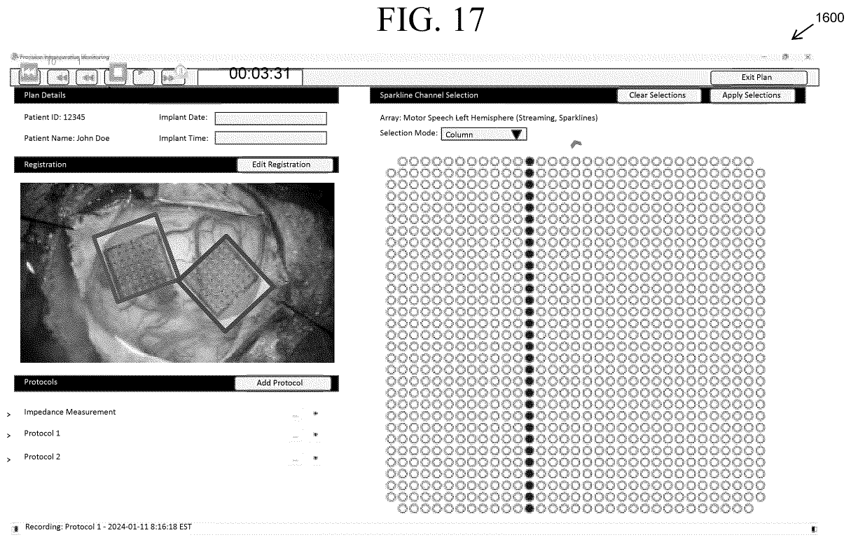

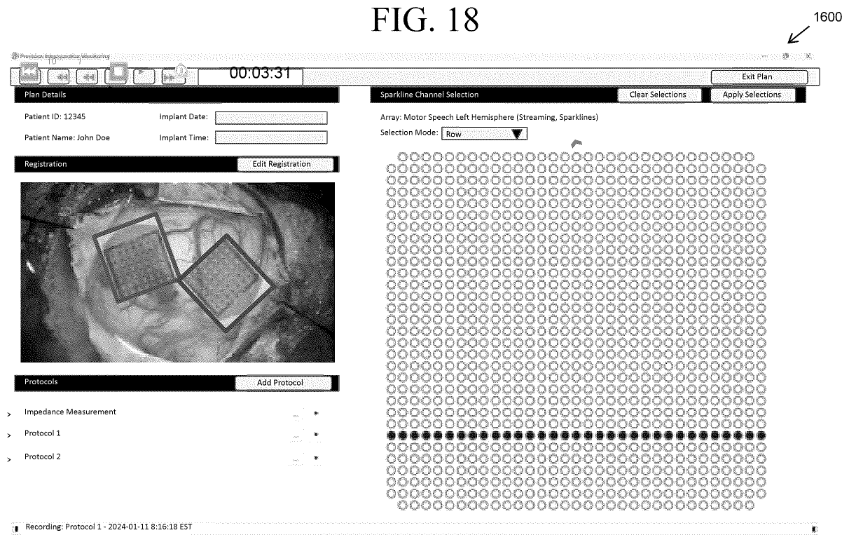

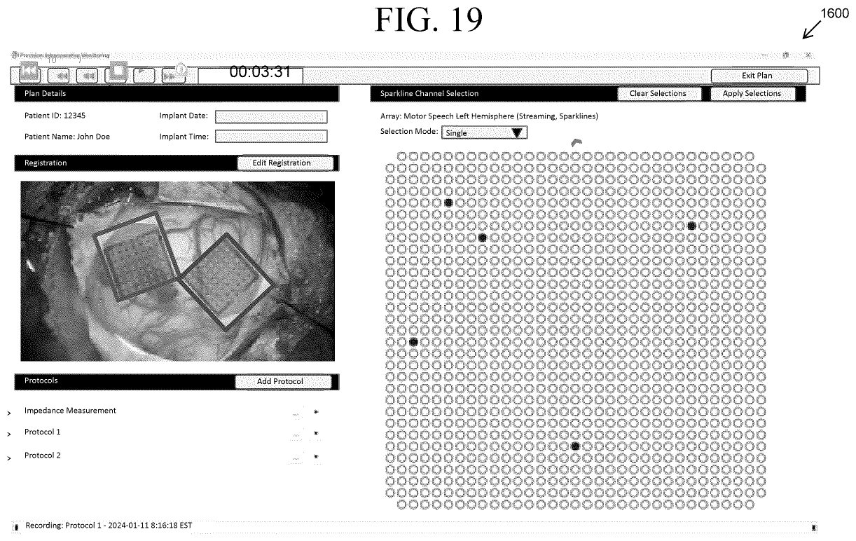

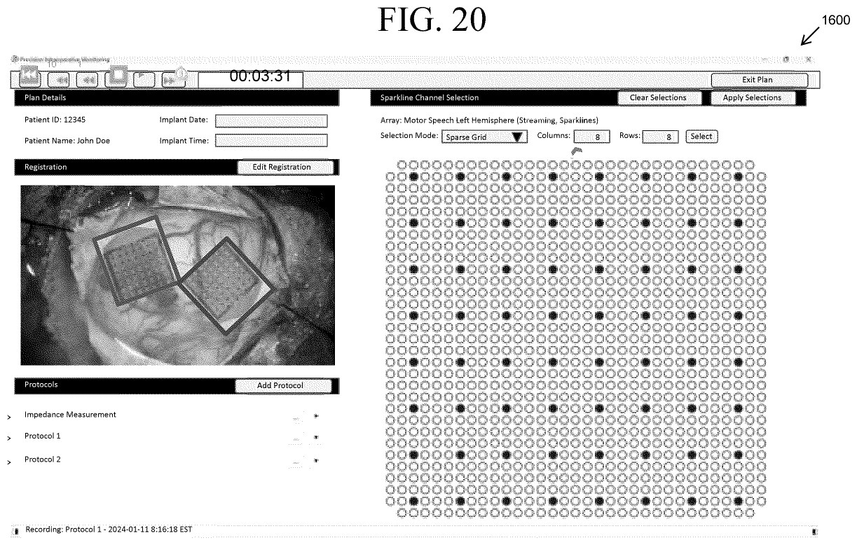

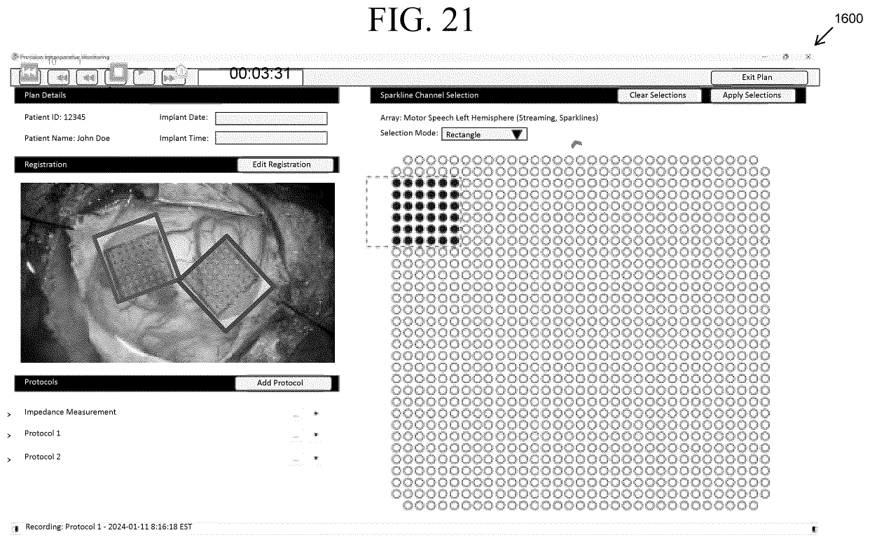

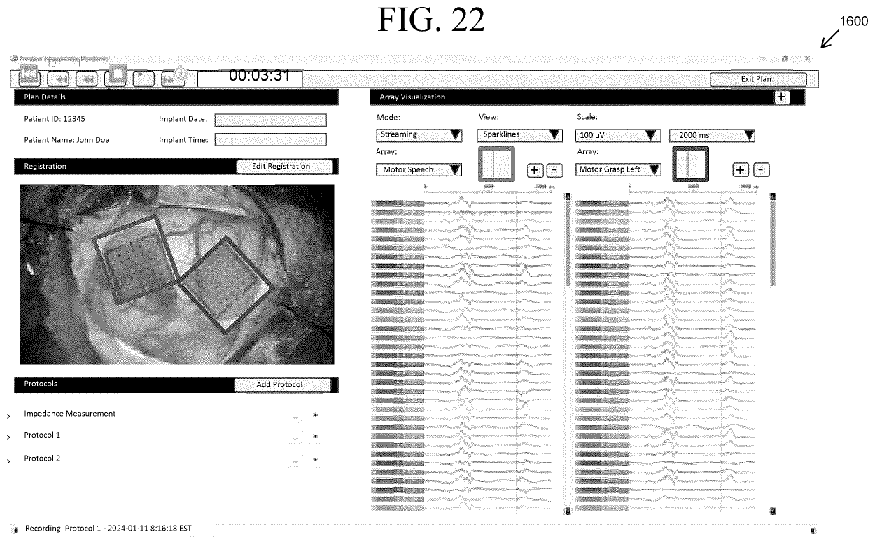

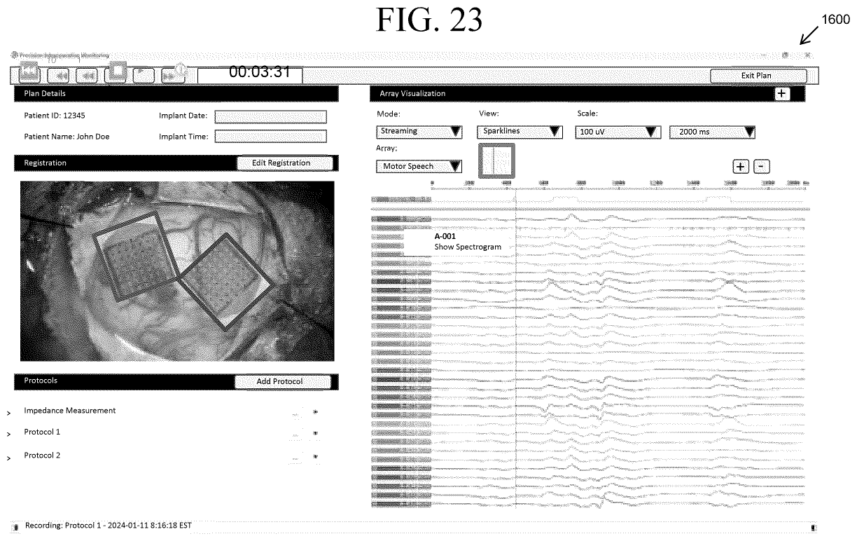

As another example, the neuronavigation system can provide sparkline visualizations 1600, as shown in FIGS. 16-23. The sparkline visualization 1600 can be used to, for example, provide guidance to assist in positioning the array accurately on the cortical surface. Further, the neuronavigation system can display real-time feedback on array placement and signal quality. The sparkline visualization 1600 can, for example, display neural signals captured across individual electrodes or groups of electrodes. In some embodiments, the neuronavigation interface can allow for surgeons to select columns of channels (FIG. 17), rows of channels (FIG. 18), individual channels (FIG. 19), grids of channels (FIG. 20), and/or various geometric arrangements of electrodes (e.g., rectangles, as shown in FIG. 21) that can be visualized using the sparkline visualization 1600. Further, sparkline visualization 1600 can be updated in real-time as new neural data is acquired, allowing for continuous monitoring of functional activity throughout the surgical procedure. As shown in FIG. 22, the sparkline visualization 1600 can further be used for surgical plan management by allowing for surgeons to select, record, and track neural signal activity across different electrode arrays separately. For example, FIG. 22 illustrates separate sparkline visualizations 1600 for arrays positioned on the cortical surface to record different tasks, each of which has individually customizable visualization features. Alternatively, the neuronavigation interface can allow for the neural activity data to be visualized in a digital and/or analog manner, as shown in FIG. 23.

By providing intuitive and responsive visualizations of functional boundaries derived from high-resolution neural recordings, the system can support more informed decision-making during neurosurgical procedures. The ability to identify and preserve eloquent cortical areas in real-time can contribute to improved surgical outcomes and reduced risk of postoperative neurological deficits.

The system can include software components for impedance measurement, noise identification, and signal quality confirmation of the electrode array. These tools can be used to verify proper contact between the electrodes and cortical tissue, identify any sources of interference, and ensure high-quality neural recordings.

Once the electrode array is positioned, the workflow can involve confirming the placement location using tracking methods provided by the neuronavigation system. This confirmation step can help ensure accurate registration between the physical array position and its representation in the neuronavigation interface. In some cases, the user interface can allow the neurosurgeon and neuronavigation representative to interact with the visualized data, adjust display parameters, and annotate important observations. The interface can provide options for selecting specific channels or regions of interest on the electrode array for more detailed analysis. The workflow can support dynamic updates and adjustments throughout the procedure. As new neural data is acquired and processed, the system can update visualizations in real-time to reflect changes in functional activity patterns or signal characteristics. This continuous feedback can allow the surgical team to adapt their approach based on the most current information available. By integrating neuronavigation capabilities with high-resolution neural recordings, the workflow can enable more precise functional mapping and guidance during neurosurgical procedures. The coordination between the neurosurgeon and neuronavigation representative, supported by intuitive user interfaces and real-time data visualization, can contribute to improved surgical outcomes and reduced risk of complications.

In some cases, the system can integrate multiple components to provide real-time surgical image guidance capabilities in a neural interface system 100. The integration of high-resolution neural recording, advanced signal processing, and neuronavigation technologies can enable comprehensive monitoring and visualization during neurosurgical procedures. The integration of the high-density electrode array with the neuronavigation system can allow for precise localization of recorded neural signals within the context of the patient's brain anatomy. The system can register the position of the electrode array relative to preoperative and intraoperative imaging data, enabling the overlay of functional information derived from neural recordings onto structural brain images.

The processed neural data can be visualized within the neuronavigation interface, providing surgeons with a comprehensive view that combines structural and functional information. This integrated display can allow for more informed decision-making during critical stages of neurosurgical procedures, such as tumor resection or epilepsy surgery. The system's ability to process and visualize high-resolution data recorded across a large number of electrode channels (e.g., 1,024 channels) in real-time can provide high spatial and temporal resolution mapping of cortical function. This detailed functional mapping can assist surgeons in identifying and preserving eloquent brain areas while maximizing the extent of resection or optimizing the placement of therapeutic interventions.

In some implementations, the system can support dynamic updating of functional visualizations throughout the surgical procedure. As new neural data is continuously acquired and processed, the displayed functional boundaries and activity patterns can be updated in real-time. This dynamic feedback can allow surgeons to monitor changes in cortical function in response to surgical manipulations or anesthesia effects. The integration of high-resolution neural recording capabilities with neuronavigation can enable new approaches to intraoperative functional mapping. For example, the system can allow for correlation between observed neural activity patterns and the effects of direct cortical stimulation, potentially providing complementary information about local brain function.

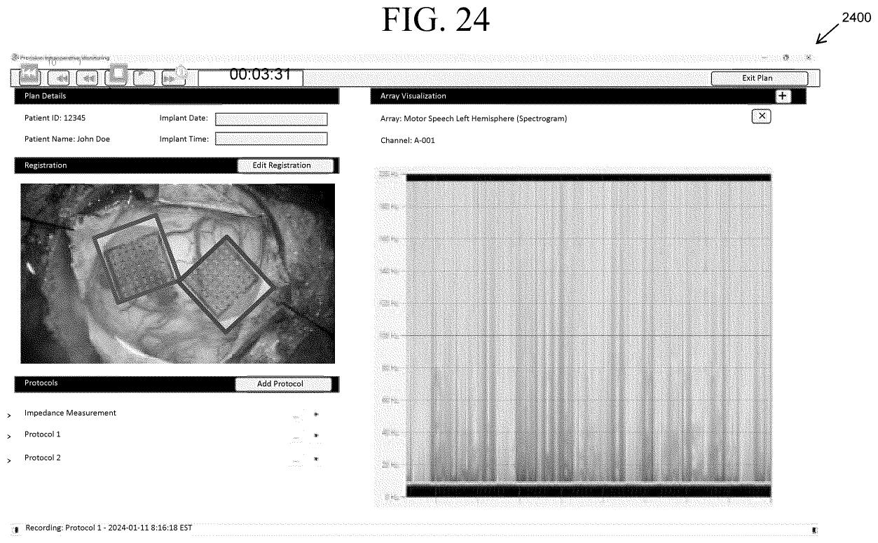

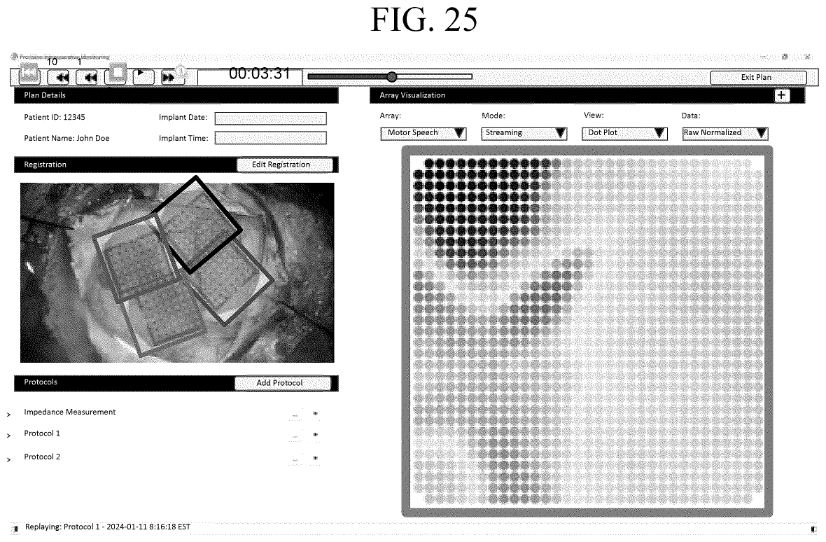

In some implementations, the system 100 can generate spectrogram visualizations to represent the frequency content of neural signals over time. For example, the system can include a spectrogram visualization 2400 where the vertical axis can represent frequency, while the horizontal axis can represent time, as shown in FIG. 24. Signal power at different frequencies can be indicated by color intensity, allowing for identification of oscillatory patterns or changes in spectral content during the recording. In some implementations, the system can provide heatmap visualizations to represent spatial patterns of neural activity. Heatmaps can use color gradients to indicate signal intensity or other derived metrics across the electrode array. This visualization mode can be particularly useful for identifying regions of elevated activity or mapping functional boundaries on the cortical surface. In some implementations, the system can provide playback and time-synchronized visualization of neural data. For example, recorded neural activity can be replayed (e.g., as shown in FIG. 25) and visualized alongside other relevant data streams. This capability can enable retrospective analysis of specific events or time periods during the surgical procedure. The system can employ various signal processing algorithms to extract meaningful features from the raw neural recordings. These can include spectral analysis techniques to quantify oscillatory activity in different frequency bands, connectivity measures to assess functional interactions between brain regions, and dimensionality reduction methods to identify dominant patterns in the high-dimensional data.

In some cases, the system can incorporate machine learning algorithms to analyze the high-dimensional neural data and extract clinically relevant features. These algorithms can assist in automatically identifying functional boundaries, detecting anomalous activity patterns, or classifying different types of neural responses observed during the procedure. The integrated system can support customizable visualization options to suit different clinical needs and user preferences. Surgeons can be able to adjust display parameters, select specific frequency bands of interest, or focus on particular spatial regions within the electrode array coverage area. By combining high-channel count neural recording, advanced signal processing, and intuitive visualization within a neuronavigation framework, the system can provide a comprehensive platform for real-time surgical guidance. This integration can support more precise and informed decision-making during neurosurgical procedures, potentially leading to improved outcomes and reduced risk of postoperative neurological deficits.

As noted above, examples have been provided of using surgical image guidance systems and processes in the context of implanting BCIs. However, that is only an illustrative use case and the present disclosure is not limited solely to the application of neurosurgical procedures for implanting BCIs. To the contrary, the surgical image guidance systems and techniques described are generally useful in a variety of different neurosurgery applications and, accordingly, the systems and processes described herein could be implemented in other applications, such as locating particular cortical regions with a level of precision that is not possible based on structural anatomy alone.

A number of implementations have been described. Nevertheless, it will be understood that various modifications can be made without departing from the spirit and scope of the disclosure. Accordingly, other implementations are within the scope of the following claims.

This disclosure is not limited to the particular systems, devices, and methods described, as these can vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the term “clinically unresponsive” means a state of unresponsiveness, which includes comatose and cognitive motor dissociation, in which the patient seems to not be able to respond appropriately to stimuli.

As used herein, the term “comatose” means a state of unresponsiveness in which the patient cannot be aroused to respond appropriately to stimuli even with vigorous stimulation and shows no brain activity attempting to respond to the stimuli.

As used herein, the term “cognitive motor dissociation” means a state of unresponsiveness in which the patient can hear and comprehend verbal commands but cannot carry out those commands due to disruption of the motor pathways downstream of the cortex and shows brain activity attempting to respond to the commands.

As used herein, the singular forms “a,” “an,” and “the” include plural references, unless the context clearly dictates otherwise. Thus, for example, reference to a “protein” is a reference to one or more proteins and equivalents thereof known to those skilled in the art, and so forth.

As used herein, the term “about” means plus or minus 10% of the numerical value of the number with which it is being used. Therefore, about 75% means in the range of 65% to 85%.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A neuronavigation system comprising:
a microelectrode array configured to be placed on a brain surface and record neural signals, the microelectrode array comprising a fiducial; and
a computer system comprising:
a processor; and
a non-transitory computer-readable medium storing instructions that, when executed by the processor, cause the computer system to:
register a position of the microelectrode array with respect to the brain surface based on the fiducial,
receive neural signals from the microelectrode array, and
generate a visualization overlaying the received neural signals on a brain image based on the registered position of the microelectrode array for display during a neurosurgical procedure.

2. The neuronavigation system of claim 1, wherein the microelectrode array comprises at least 1,000 electrode channels.

3. The neuronavigation system of claim 1, wherein the instructions further cause the computer system to:
register a position of the microelectrode array relative to the brain image.

4. The neuronavigation system of claim 3, wherein registering the position of the microelectrode array comprises using optical or electromagnetic tracking.

5. The neuronavigation system of claim 1, wherein the instructions further cause the computer system to:
processing the neural signals to generate a spectral analysis on the neural signals to identify oscillatory patterns.

6. The neuronavigation system of claim 1, wherein the visualization comprises a heatmap overlay indicating levels of neural activity across the brain surface.

7. The neuronavigation system of claim 1, wherein the instructions further cause the computer system to:
update the visualization in real-time as new neural signals are received from the microelectrode array.

8. A computer system communicably connectable to a microelectrode array configured to be placed on a brain surface and record neural signals, the microelectrode array comprising a fiducial, the computer system comprising:
a processor; and
a non-transitory computer-readable medium storing instructions that, when executed by the processor, cause the computer system to:
register a position of the microelectrode array with respect to the brain surface based on the fiducial,
receive neural signals from the microelectrode array, and
generate a visualization overlaying the received neural signals on a brain image based on the registered position of the microelectrode array for display during a neurosurgical procedure.

9. The computer system of claim 8, wherein the microelectrode array comprises at least 1,000 electrode channels.

10. The computer system of claim 8, wherein the instructions further cause the computer system to:
processing the neural signals to generate a spectral analysis on the neural signals to identify oscillatory patterns.

11. The computer system of claim 8, wherein the visualization comprises a heatmap overlay indicating levels of neural activity across the brain surface.

12. The computer system of claim 8, wherein the instructions further cause the computer system to:
register a position of the microelectrode array relative to the brain image using optical or electromagnetic tracking.

13. The computer system of claim 8, wherein the instructions further cause the computer system to:
update the visualization in real-time as new neural signals are received from the microelectrode array.

14. The computer system of claim 8, wherein the instructions further cause the computer system to:
generate a spectrogram visualization representing frequency content of the neural signals over time.

15. A method for a microelectrode array configured to be placed on a brain surface and record neural signals, the microelectrode array comprising a fiducial, the method comprising:
registering, by a computer system, a position of the microelectrode array with respect to the brain surface based on the fiducial,
receiving, by the computer system, neural signals recorded by a microelectrode array placed on a brain surface;
generating, by the computer system, a visualization overlaying the received neural signals on a brain image based on the registered position of the microelectrode array; and
outputting, by the computer system, the visualization for display on a neuronavigation interface during a neurosurgical procedure.

16. The method of claim 15, wherein the microelectrode array comprises at least 1,000 electrode channels.

17. The method of claim 15, further comprising:
processing, by the computer system, the neural signals to perform spectral analysis to identify oscillatory patterns.

18. The method of claim 15, wherein the visualization comprises a heatmap overlay indicating levels of neural activity across the brain surface.

19. The method of claim 15, further comprising:
registering, by the computer system, a position of the microelectrode array relative to the brain image using optical or electromagnetic tracking.

20. The method of claim 15, further comprising:
updating, by the computer system, the visualization in real-time as new neural signals are received from the microelectrode array.
